(19) United States
(12) Patent Application Publication
(10) Pub. No.: US 2003/0199887 A1
(43) Pub. Date: Oct. 23, 2003

(54) Filamentous embolization device and method of use

(76) Inventors: David Ferrera, Manhattan Beach, CA (US); Brian J. Cox, Laguna Niguel, CA (US); George R. Greenc JR., (US)

Correspondence Address: OPPENHEIMER WOLFF & DONNELLY LLP, 840 NEWPORT CENTER DRIVE, SUITE 700, NEWPORT BEACH, CA, 92660, US

(21) Appl. No.: 10/128,917

(22) Filed: Apr. 23, 2002

(51) Int. Cl. A61B 17/08

(52) U.S. Cl. 606/151

(57) ABSTRACT

The present invention is directed to porous or textured embolization devices which are capable of being delivered to the situs of a vascular dysfunction through a delivery device. One embodiment of the embolization device comprises, in general, a resilient material thread having a first relaxed shape and a second stretched shape, wherein the first relaxed shape forms a space-filling body, and wherein the second stretched shape forms a linear body, and one or more surface irregularities formed on the material thread. In one embodiment, the one or more surface irregularities formed on the material thread may provide a porous material thread and encourage tissue in-growth. In another embodiment, the one or more surface irregularities formed on the material thread may provide a textured material thread capable of promoting tissue in-growth.

### BACKGROUND OF THE INVENTION

The mammalian circulatory system is comprised of a heart, which acts as a circulatory pump, and a system of blood vessels which transports the blood to various points throughout the body. The blood vessels may develop a variety of vascular disabilities or dysfunctions due to the force exerted by the flowing blood on the blood vessels. One common vascular dysfunction is known as an aneurysm. Vascular aneurysms are formed as a result of the weakening of the wall of a blood vessel, typically resulting in the ballooning of the vessel wall. Commonly, aneurysms form at vascular junctions or areas of increased intra-vascular flow pressure.

Aneurysms have been known to form in a variety of locations though the body, including, for example, the brain, the abdomen, and throughout the circulatory system. For example, one study estimates that as many as 400,000 people in the United States may have an intracranial aneurysm of significant size, while more than 25,000 people suffer a rupture of an intracranial aneurysm annually. The rupture of the brain aneurysm may result in, a subarachnoid hemorrhage, wherein blood from the hemorrhage is trapped between the brain and the brain's covering. The increasing volume of blood trapped within the confined space exerts pressure on the brain and could result in a stroke, brain damage, or death. Currently, there is a less than complete understanding of the processes leading to the formation of aneurysms throughout the body, although several factors increasing the likelihood of formation have been identified.

In response, several surgical techniques for treating aneurysms have been developed. Initially, an aneurysmectomy was required to repair the dysfunctional tissue. The aneurysmectomy procedure requires the surgeon to gain access to the aneurysm, excise the aneurysm, and replace the void with a prosthetic graft. Because this is a major surgical undertaking, the mortality rate of the procedure is relatively high. Commonly, the aneurysmectomy procedure is unavailable to patients with severe coronary or cerebral arteriosclerosis, severe restrictive pulmonary disease, and significant renal disease or other complicating factors.

An alternate method of treating cerebral aneurysms is called ‘microsurgical clipping’. During a ‘clipping’ procedure, the surgeon gains access to the site of the aneurysm. Thereafter, the surgeon places an aneurysm clip across the neck of the aneurysm thereby occluding the aneurysm from the blood flow. The deprivation of blood flow to the aneurysm results in the ballooning tissue decreasing in size over time. While this procedure has proven successful in the past, several shortcoming of the clipping procedure have been identified. For example, additional aneurysms have been known to form on the area of the neck of the aneurysm proximal to the aneurysm clip.

In response to the shortcomings of the aneurysmectomy and the clipping procedures, less invasive methods of treatment have been developed. The vascular embolization procedure requires the insertion of an artificial vaso-occlusive device within the ballooning tissue forming the aneurysm, thereby reducing the blood flow to the aneurysm. The reduction in blood flow results in hemostasis and the formation of a clot within the aneurysm or proximal to the aneurysm neck. The aneurysm decreases in size over time as a result of the hemostasis. The vascular embolization procedure requires the surgeon to advance a micro-catheter to a location within the aneurysm or proximal the opening of the aneurysm. Thereafter, a biologically-compatible vaso-occlusive device is deposited therein. Typical vaso-occlusive devices and materials include platinum micro-coils, hog hair, microfibrillar collagen, various polymeric agents, material suspensions, and other space filling materials. While embolization procedures using the aforementioned vaso-occlusive devices have proven successful, several shortcomings of these devices have been identified. For example, maintaining the vaso-occlusive devices within the aneurysm has proven problematic in the past. Blood flow through an otherwise functional blood vessel may be compromised should the vaso-occlusive device migrate from the aneurysm during or following implantation, possibly resulting in a vascular embolism. Ideally the vaso-occlusive device would occupy essentially the entire volume of the ballooning tissue forming the aneurysm, thereby eliminating or substantially reducing the volume within the aneurysm capable of receiving blood flow. Over time, the pressure exerted on current vaso-occlusive devices by the blood flowing through the blood vessel may result in the device becoming repositioned or compacted within the aneurysm, thereby forming cavities or areas capable of receiving blood flow therein and resulting in the less than satisfactory embolization of the aneurysm.

In light of the foregoing, there is a long felt need for a vaso-occlusive device and method of use capable of embolizing an aneurysm without significantly affecting blood flow through the blood vessel.

### BRIEF SUMMARY OF THE INVENTION

The present invention provides a filamentous embolization device capable of being inserted into a body cavity or vascular anomaly. For example, the present invention may be inserted into a vascular aneurysm and used to reduce the flow of blood thereto. The present invention includes a material substrate having one or more surface irregularities formed thereon or therein which promote tissue in-growth or endothelialization. The filamentous embolization device of the present invention enables a user to treat an aneurysm formed through the patient's body from a remote location.

In one embodiment, the filamentous embolization device of the present invention comprises a resilient material thread having a first relaxed shape and a second stretched shape, and includes one or more surface irregularities formed thereon or therein. The material thread is capable of forming a space-filling body in the first relaxed shape and a linear body in the second stretched shape.

In another embodiment, the filamentous embolization device of the present invention comprises a resilient material substrate having one or more porous elements positioned thereon. The material substrate is capable of forming a first relaxed shape, thereby providing a space-filling body. In addition, the material substrate is capable of forming a second stretched shape, wherein the second stretched shape forms a linear body to facilitate the delivery and insertion of the embolization device into a vascular dysfunction.

In yet another aspect, the present invention discloses a method of repairing a vascular dysfunction and includes accessing a site of a vascular dysfunction in vivo, providing an embolization device having one or more surface irregularities formed thereon, delivering the embolization device into the vascular dysfunction, and promoting tissue in-growth with the embolization device.

In yet another aspect, the present invention discloses a method of repairing a vascular dysfunction which comprises accessing a site of a vascular dysfunction in vivo, providing an embolization device comprising a material substrate having one or more porous elements positioned thereon, delivering the embolization device into the vascular dysfunction, and promoting tissue in-growth with the embolization device.

Other objects, features, and advantages of the present invention will become apparent from a consideration of the following detailed description.

### BRIEF DESCRIPTION OF THE DRAWINGS

The apparatus of the present invention will be explained in more detail by way of the accompanying drawings, wherein:

FIG. 1 shows a perspective view of an aneurysm formed on a blood vessel of a patient;

FIG. 2 shows a perspective view of the porous embolization device of the present invention being applied to an aneurysm formed on a blood vessel of a patient with a delivery device;

FIG. 3a shows a side view of the porous embolization device of the present invention, a portion of which has assumed a first relaxed shape and a portion of which remains in a second stretched insertion shape;

FIG. 3b shows a side view of an embodiment of the porous embolization device of the present invention wherein the embolization device is formed into a helical or coiled structure;

FIG. 4 shows a cross-sectional view of the porous embolization device of the present invention formed into a helical or coiled structure;

FIG. 5 shows a perspective view of another embodiment of the porous embolization device of the present invention wherein the embolization device is formed into a cylindrical structure;

FIG. 6 shows a detailed view of the external surface of the porous embolization device of the present invention;

FIG. 7 shows a cross-sectional view of another embodiment of the helical embolization device of the present invention wherein the external surface of the embolization device is textured;

FIG. 8 shows a perspective view of another embodiment of the textured embolization device of the present invention wherein the embolization device is formed into a cylindrical structure;

FIG. 9 shows a detailed view of the external surface of the textured embolization device of the present invention;

FIG. 10 shows a perspective view of the another embodiment of the embolization device of the present invention attached to a delivery device;

FIG. 11 shows a cross-sectional view of the another embodiment of the embolization device of the present invention having at least one porous element and at least one visualization element thereon;

FIG. 12 shows a detailed view of the at least one porous element of the embolization device of the present invention;

FIG. 13 shows a cross-sectional view of the another embodiment of the embolization device of the present invention having at least one porous element thereon;

FIG. 14 shows a cross-sectional view of another embodiment of the embolization device of the present invention having at least one porous element and at least one helical or coiled member located on a material substrate; and

FIG. 15 shows a side view of another embodiment of the embolization device of the present invention having at least one porous element positioned on an intermittently coiled substrate.

### DETAILED DESCRIPTION OF THE INVENTION

Disclosed herein is a detailed description of various illustrated embodiments of the present invention. This description is not to be taken in a limiting sense, but is made merely for the purpose of illustrating the general principles of the present invention. The section titles and overall organization of the present detailed description are for the purpose of convenience only and are not intended to limit the present invention.

The filamentous embolization device of the present invention generally comprises a material substrate having one or more surface irregularities formed therein or thereon. Exemplary surface irregularities include openings, channels, or textures formed on or into the material substrate, thereby providing a porous or textured filamentous embolization device capable of promoting tissue in-growth, endothelialization, or otherwise imparting therapeutic effects on tissue. Those skilled in the art will appreciate that the surface irregularities may be formed in a variety of sizes as desired. For example, in one embodiment the surface irregularities may have a length or diameter within the range of about 0.005 mm to about 0.2 mm. In yet another embodiment, the surface irregularities may have a height or depth of at least about 0.05 mm. Generally, the filamentous embolization device of the present invention is used to occlude or otherwise restrict the ability of a blood flow from entering an aneurysm formed on a blood vessel. While the present invention may be used to repair a variety of aneurysms formed at various locations throughout the body, the present invention is particularly well suited to fill space within a body cavity or otherwise impart therapeutic effects on tissue located proximate thereto. In addition, the present invention may incorporate at least one material capable of imparting therapeutic effects on tissue located proximate thereto, Exemplary therapeutic material may include, for example, bio-active agents, proteins, peptides, marking agents, vascular endothelial growth factors (VEGF), basic fibroblast growth factors (bFGF), transforming growth factors-β (TGF-β) Hyaluronan derivatives [2,2], paracyclophanes, agenine-glycerine-aspartic acid (RGD), platelet derived growth factor (PDGF), thrombospondin 1 (TSP1), alginate, collagen, glycoprotein, glycosaminoglycan, endotehlial cells, tissue submucosa cells, tissue mucosa cells, and intestinal submucosa cells (SIS).

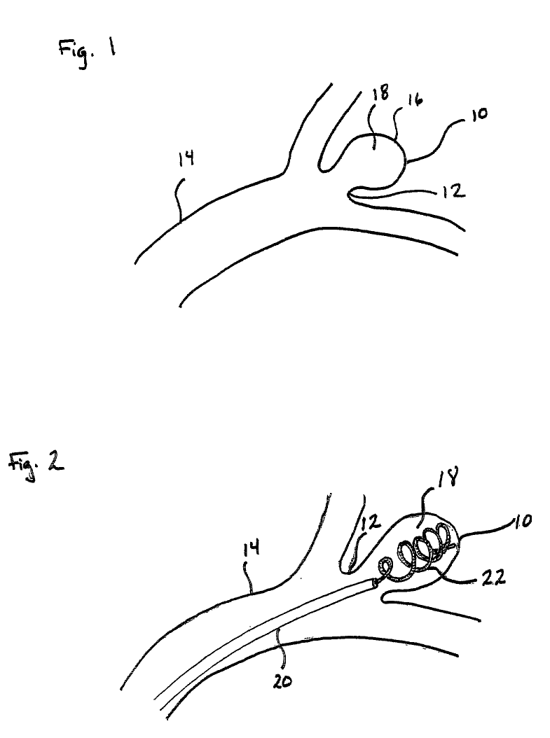

Aneurysms form as a result of outward pressure applied to a diseased or damaged blood vessel wall by blood flowing within the vessel. Over time, the outwardly applied pressure causes a weakened section of vessel tissue to balloon outwardly, thereby forming an aneurysmal cavity on the blood vessel. FIG. 1 shows an aneurysm 10 comprising a neck portion 12 in communication with a blood vessel 14 and having a dome portion 16 defining an aneurysmal cavity 18. Those skilled in the art will appreciate that FIG. 1 illustrates an exemplary vascular aneurysm and is not intended to limit the scope or intent of the present invention or its environment of use. For example, the present invention may be used to fill, treat or otherwise impart therapeutic effects to vascular anomalies or tissue formed throughout the body.

In one embodiment, a porous filamentous embolization device of the present invention is used to embolize aneurysms formed throughout the body. FIG. 2 shows an aneurysm 10 having a neck portion 12 in communication with a blood vessel 14. A delivery device 20, located within the blood vessel 14, is shown depositing a porous embolization device into the aneurysmal cavity 18 forming the aneurysm 10. Those skilled in the art will appreciate that the porous embolization device 22 of the present invention may be applied to an aneurysm 10 formed on a blood vessel in a variety of ways, including, without limitation, conventional surgical techniques and minimally invasive surgical techniques utilizing catheters of various sizes, including microcatheters, and other ways generally known in the art of minimally invasive surgery including delivery devices having electrolytically severable tips.

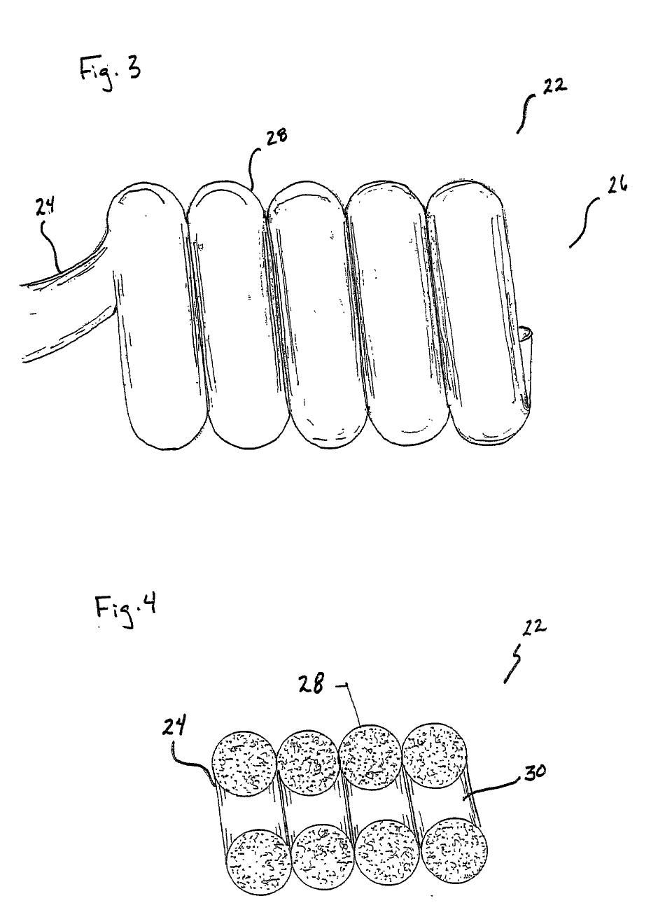

FIGS. 3a-5 show embodiments of the porous embolization device of the present invention. As shown in FIGS. 3a-5, the embolization device 22 comprises a resilient material thread 24 capable of being formed into a random, convoluted, or predetermined first relaxed shape and a second stretched insertion shape. Exemplary first relaxed shapes include, for example, helical, spherical, ovoidal, conical shapes, or other space-filling shapes. The second stretched insertion shape facilitates delivery of the embolization device 22 through a catheter or other delivery device. For example, FIG. 3a shows an embolization device 22 of the present invention positioned on a delivery device 20 being applied to an aneurysm 10. As shown in FIG. 3a, a first portion of the embolization device 22 has been disengaged from the delivery device 20 and has assumed a first relaxed shape 23a, while a second portion of the embolization device 22 remains positioned on the delivery device 20 in a second stretched insertion shape 23b.

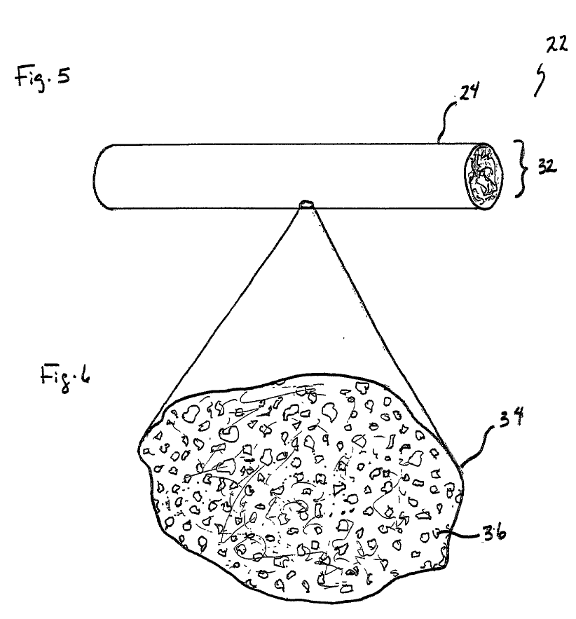

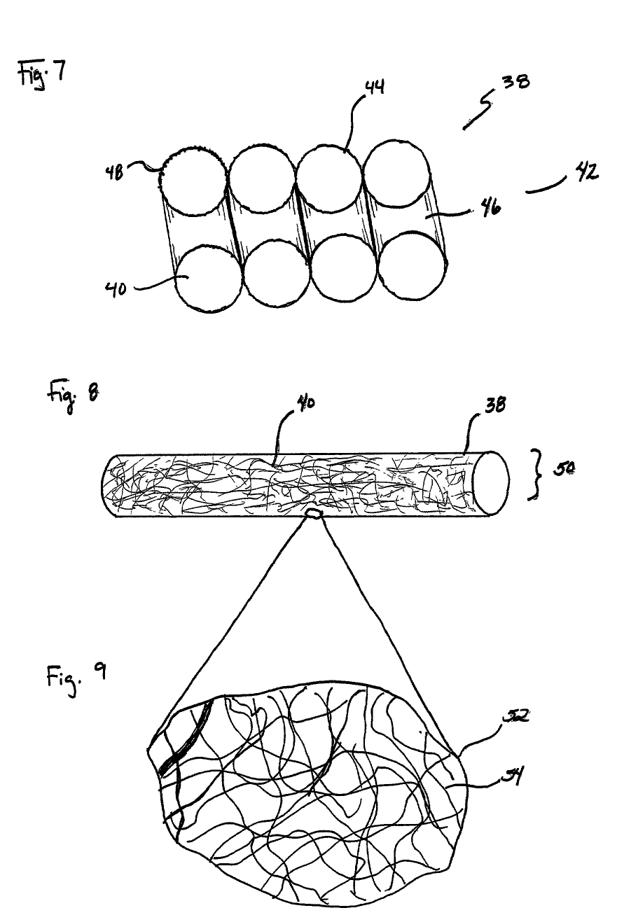

As illustrated in FIGS. 3a-4, the material thread 24 may comprise at least one helical winding 28 or may otherwise form a helical structure 26, wherein the material thread windings 28 define an internal void 30. FIG. 5 shows an alternate embodiment wherein the material thread 24 of the porous embolization device 22 forms a cylindrical structure 32. Those skilled in the art will appreciate that the material thread 24 may be manufactured in a variety of diameters and lengths from a variety of biologically-compatible materials, including, for example, platinum, gold, tantalum, titanium, stainless steel, tungsten, Nitinol, shape memory alloys, polyurethane, polyvinyl alcohol, polyester, polytetrafluoroethylene, silicone, acrylic, or other suitable material. In yet another embodiment, the material thread 24 may incorporate radio-opaque or echogenic materials, thereby enabling the surgeon to precisely position the embolization device 22 within a blood vessel or other hollow organ. Those skilled in the art will appreciate that the material thread 24 may be chemically doped or impregnated with at least one drug, bio-active compound, or growth-promoting material to encourage tissue growth or impart other therapeutic benefits to the tissue located near the material thread 24. For example, the present invention may incorporate various bio-active agents, proteins, peptides, marking agents, vascular endothelial growth factors (VEGF), basic fibroblast growth factors (bFGF), transforming growth factors-β (TGF-β) Hyaluronan derivatives [2,2], paracyclophanes, agenine-glycerine-aspartic acid (RGD), platelet derived growth factor (PDGF), thrombospondin 1 (TSP1), alginate, collagen, glycoprotein, glycosaminoglycan, endotehlial cells, tissue submucosa cells, tissue mucosa cells, and intestinal submucosa cells (SIS).

FIG. 6 shows a detailed view of the external surface of the embolization device 22. As shown in the detailed view 34, the material thread 24 may include one or more pores or openings 36 formed thereon. In one embodiment, the one or more pores or openings 36 may be of variable size or shape formed in the material thread 24. In an alternate embodiment, the one or more pores or openings 36 may be of substantially identical sizes or shapes formed in the material thread 24. The one or more pores or openings 36 may terminate within the material thread 24 or, in the alternative, the one or more pores or openings 36 may be in communication with continuous pathways which traverse the material thread 24. As shown in FIG. 6 the one or more pores or openings 36 form an irregular surface on material thread 24. When inserted into a vascular aneurysm the irregular surface formed by the one or more pores or openings 36 permits the vascular tissue forming the aneurysm to engage the porous embolization device and facilitates tissue in-growth or endothelialization. Those skilled in the art will appreciate that the one or more pores or openings 36 may be capable of receiving and storing at least one drug or growth-promoting material therein to encourage tissue growth or impart other therapeutic benefits to targeted vascular tissue.

FIGS. 7-9 show various views of alternate embodiments of the embolization device of the present invention. Like the previous embodiments, the embolization device 38 of the present embodiments comprises a resilient material thread 40 capable of being formed into a random, convoluted, or predetermined first relaxed shape and a second stretched insertion shape. Exemplary first relaxed shapes include, for example, helical, spherical, ovoidal, conical shapes, or other space-filling shapes. The second stretched insertion shape facilitates delivery of the embolization device 38 through a catheter or other delivery device. As illustrated in FIG. 7, the material thread 40 may comprise at least one helical winding 44 or may otherwise form a helical structure, wherein the material thread windings 44 define an internal void 46. In these embodiments, the external surface 48 of the material thread 40 may be textured, channeled, or may otherwise incorporate one or more surface irregularities thereon to facilitate tissue in-growth or endothelialization. FIG. 8 shows another embodiment of a textured embolization device 38 of the present embodiment, wherein the embolization device 38 is formed into a cylindrical structure 50. FIG. 9 shows a detailed view of a portion of a textured embolization device 38 of the present invention. As shown in the detailed view 52, the material thread 40 includes at least one texture element, channel, or other surface irregularity 54 thereon. In one embodiment, the at least one texture element, channel, or other surface irregularity 54 may be of variable size or shape, randomly formed in the material thread 40. In an alternate embodiment, the at least one texture element, channel, or other surface irregularity 54 may be of substantially uniform size or shape formed in the material thread 40. An exemplary surface irregularity may include, for example, a groove, bump, barb, matrice, fenestration, notched, bump, or tooth. The at least one texture element, channel, or other surface irregularity 54 may be capable or receiving or storing at least one drug or growth-promoting material therein to encourage tissue growth or impart other therapeutic benefits to targeted vascular tissue. Like the previous embodiments, the textured embolization device 38 of the present embodiments may be manufactured in a variety of diameters and lengths from a variety of biologically-compatible materials, including, for example, platinum, gold, tantalum, titanium, stainless steel, tungsten, Nitinol, shape memory alloys, polyurethane, polyvinyl alcohol, polyester, polytetrafluoroethylene, silicone, acrylic, or other suitable material. In addition, the material thread 40 may incorporate fluoroscopic materials, x-ray materials, magnetic resonant materials, ultrasonic imaging materials, radio-opaque materials, or echogenic materials, thereby enabling the surgeon to precisely position the embolization device 38 within a blood vessel or other hollow organ. Those skilled in the art will appreciate that the material thread 40 may be chemically doped or impregnated with a drug or growth-promoting material to encourage tissue growth or impart other therapeutic benefits to the tissue located near the material thread 40.

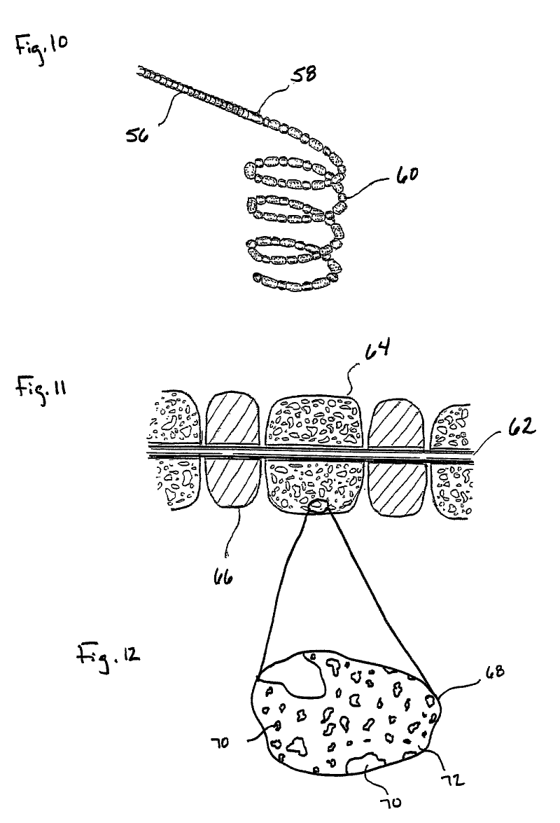

FIGS. 10-12 show yet another embodiment of the present invention. FIG. 10 shows a delivery device 56 having a detachment tip 58 in communication with a visualizable porous embolization device 60 of the present invention. As shown in FIG. 11, the visualizable porous embolization device 60 comprises a resilient material substrate 62 having at least one porous element 64 and at least one visualization element 66 positioned thereon or otherwise in communication therewith. Like the previous embodiments, the material substrate 62 may be capable of being formed into a random, convoluted, or predetermined first relaxed shape and a second stretched insertion shape. Exemplary first relaxed shapes include, for example, helical, spherical, ovoidal, conical shapes, or other space-filling shapes. The second stretched insertion shape facilitates delivery of the embolization device 62 through a catheter or other delivery device. In addition, the material substrate 62 may be manufactured from a variety of materials, including, without limitation, platinum, gold, tantalum, titanium, stainless steel, tungsten, Nitinol, shape memory alloys, polyurethane, polyvinyl alcohol, polyester, polytetrafluoroethylene, silicone, acrylic, or other suitable material. The material thread 40 may incorporate radio-opaque or echogenic materials or agents. The at least one visualization element may be manufactured from a variety of biologically compatible materials which are highly visible to a variety of imaging devices. Exemplary imaging devices may include, without limitation, fluoroscopic devices, x-ray devices, magnetic resonance devices, and ultrasonic imaging devices. As such, the at least one visualization element may include radio-opaque materials or echogenic materials incorporated therein. In some embodiments, the porous elements 64 may comprise an expansible material that will expand either by hydrophilic hydration or from the pores being filled by blood, or both. In other embodiments the porous elements may comprise non-expansible material.

FIG. 12 shows a detailed view of the at least one porous element 64 of the present embodiment. As shown in the detailed portion 68, the at least one porous element 64 includes one or more openings 70 formed in a porous substrate 72. As shown, the one or more openings 70 may be of variable size and distribution, or, in the alternative, may be of uniform size and positioning on the porous substrate 72. The one or more openings 70 may facilitate tissue in-growth or endothelialization and may include a drug or growth-promoting material to encourage tissue growth or impart other therapeutic benefits to the tissue located near the porous substrate 70. Those skilled in the art will appreciate that the at least one porous element 64 may be manufactured in a variety of sizes, shapes, and diameters as desired. The at least one porous element 64 may be manufactured from a variety of materials, including, without limitation, platinum, gold, tantalum, titanium, stainless steel, tungsten, Nitinol, polyurethane, polyvinyl alcohol, polyester, polytetrafluoroethylene, silicone, acrylic, or other suitable material, and may include radio-opaque or echogenic materials or agents. As disclosed above, the materials used to make the at least one porous element may be expansible or non-expansible.

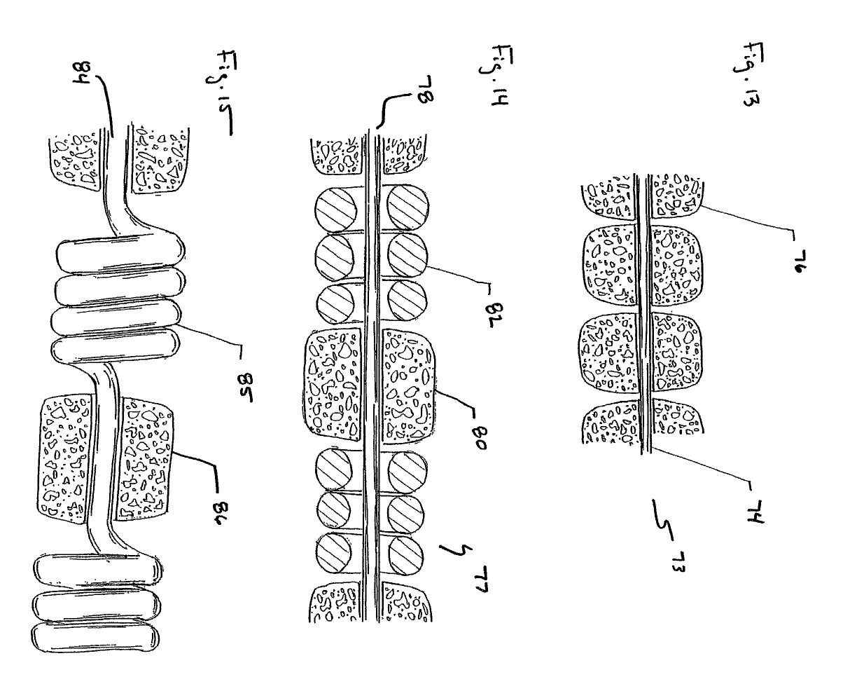

FIGS. 13-15 show various alternate embodiments of the present invention. Like the previous embodiments, the embolization devices shown in FIGS. 13-15 may be capable of being formed into a random, convoluted, or predetermined first relaxed shape and a second stretched insertion shape. Exemplary first relaxed shapes include, for example, helical, spherical, ovoidal, conical shapes, or other space-filling shapes. The second stretched insertion shape facilitates delivery of the embolization devices of the present embodiments through a catheter or other delivery device. As shown in FIG. 13, the embolization device 73 may comprise a material substrate 74 having at least one porous element 76 positioned thereon. FIG. 14 shows another embodiment wherein the embolization device 77 comprises material substrate 78 having at least one porous element 80 and at least one coil element positioned thereon. FIG. 15 shows yet another embodiment wherein an intermittently coiled substrate 84 includes at least one coiled section 85 and has at least one porous element 86 disposed thereon. Similar to the previous embodiments, the present embodiments shown in FIGS. 13-15 may be manufactured from a from a variety of materials, including, without limitation, platinum, gold, tantalum, titanium, stainless steel, tungsten, Nitinol, shape memory alloys, polyurethane, polyvinyl alcohol, polyester, polytetrafluoroethylene, silicone, acrylic, or other suitable material, and may include radio-opaque or echogenic materials or agents. Those skilled in the art will appreciate that the substrates of the present embodiments shown in FIGS. 13-15 may be porous, textured, or may otherwise incorporate one or more surface irregularities thereon to increase tissue in-growth or endothelialization.

The present invention further discloses a method of treating vascular aneurysms. In the one embodiment, the present invention discloses a method of inserting an embolization device into a blood vessel, advancing the embolization device to a location proximate to a vascular aneurysm, and inserting the device to the aneurysm. Those skilled in the art will appreciate that the embolization device of the present invention may be delivered to a situs in vivo in a variety of manners, including, for example, through micro-catheters or catheters having electrostatically separable tips. FIGS. 2 and 3a show an exemplary embodiment 22 of the present invention being delivered into an aneurysm 10 using a delivery device 20.

In practice, the surgeon positions an embolization device 22 on or within a delivery device 20. As shown in FIG. 3a, prior to positioning the embolization device 22 of the delivery device 20 the embolization device 22 is formed into the second stretched insertion shape 23b, thereby permitting the embolization device 22 to be retained within the delivery device 20. Thereafter, a first incision is made proximate a blood vessel and a guidewire may be inserted therein. Commonly, the guidewire will enter the circulatory system through the femoral artery, the femoral vein, the jugular vein, the carotid artery, or a similar blood vessel. If used, the guidewire may then be directed through the circulatory system to a location proximate to the aneurysm and, thereafter, made to exit the body through a remote exit point. The delivery device 20 having embolization device 22 attached thereto or positioned therein is introduced into the body. The delivery device 20 may be attached to the guidewire, if present, and advanced along the guidewire to a position proximate to the aneurysm 10. Typically, visualization methods, such as fluoroscopy, ultrasound visualization, or echogenic visualization are utilized to precisely position the delivery device 20 near the aneurysm neck 12 or within the aneurysm 10. Once positioned, the distal end of the embolization device 22 is inserted into the aneurysm 10. Thereafter, the embolization device 22 is controllably dispensed from the delivery device 20 into the aneurysm 10, thereby filling the aneurysmal cavity 18 with the embolization device 22. When the aneurysmal cavity 18 is sufficiently filled by the embolization device 22, the embolization device 22 is detached from the delivery device 20. For example, in one embodiment an electrostatically separable tip of the delivery device 20 is actuated, thereby separating the proximal portion of the embolization device 22 from the delivery device 20. An exemplary electrostatically separable tip is disclosed in U.S. Pat. No. 5,122,136, issued on Jun. 12, 1992, entitled “Endovascular Electrolytically Detachable Guidewire Tip for the Electroformation of Thrombus in Arteries, Veins, Aneurysms, Vascular Malformations and Arteriovenous Fistulas,” which is incorporated by reference in its entirety herein.

Once the embolization device 22 is separated from the delivery device 20, the delivery device 22 and guidewire, if used, are removed from the body. Insertion of the embolization device 20 into the aneurysm results in the embolization device 22 assuming a first relaxed, convoluted, or predetermined shape 23a, thereby reducing or eliminating the blood flow to the aneurysm. The surface irregularity formed on the embolization device of the present invention results in increased tissue in-growth or endothelialization, thereby increasing the therapeutic benefits of the present invention over prior art devices.

In closing it is understood that the embodiments of the invention disclosed herein are illustrative of the principles of the invention. Other modifications may be employed which are within the scope of the invention. Accordingly, the present invention is not limited to that precisely as shown and described in the present invention.

What is claimed is:

1. A device for repairing vascular dysfunction or imparting a therapeutic effect on tissue in vivo, comprising:
a resilient material thread having a first relaxed shape and a second stretched shape, wherein said first relaxed shape forms a space-filling body, and wherein said second stretched shape forms a linear body; and
one or more surface irregularities formed on a surface of said material thread.

2. The device of claim 1 wherein said material thread may be manufactured from at least one biologically compatible material selected from the group consisting of platinum, gold, tantalum, titanium, stainless steel, tungsten, Nitinol, shape memory alloys, polyurethane, polyvinyl alcohol, polyester, polytetrafluoroethylene, silicone, and acrylic.

3. The device of claim 1 wherein said first relaxed shape is a predetermined shape selected from the group consisting of helical, spherical, ovoidal, and conical shapes.

4. The device of claim 1 wherein said first relaxed shape is a random shape.

5. The device of claim 1 wherein said second stretched insertion shape is capable of being inserted into a catheter.

6. The device of claim 1 wherein said material thread comprises at least one helical winding.

7. The device of claim 1 wherein said material thread comprises a cylindrical structure.

8. The device of claim 1 wherein said one or more surface irregularities are selected from the group consisting of openings, holes, texture members, grooves, bumps, barbs, matrices, fenestrations, notched, bumps, and teeth.

9. The device of claim 1 wherein said one or more irregularities are in communication with pathways formed in said material thread.

10. The device of claim 1 wherein said embolization device incorporates at least one material selected from the group consisting of bio-active agents, proteins, peptides, marking agents, vascular endothelial growth factors (VEGF), basic fibroblast growth factors (bFGF), transforming growth factors-β (TGF-β) Hyaluronan derivatives [2,2], paracyclophanes, agenine-glycerine-aspartic acid (RGD), platelet derived growth factor (PDGF), thrombospondin 1 (TSP1), alginate, collagen, glycoprotein, glycosaminoglycan, endotehlial cells, tissue submucosa cells, tissue mucosa cells, and intestinal submucosa cells (SIS).

11. The device of claim 1 wherein said material thread incorporates at least visualization material therein.

12. The device of claim 1 wherein said visualization material is selected from the group consisting of fluoroscopic materials, x-ray materials, magnetic resonant materials, ultrasonic imaging materials, radio-opaque materials, and echogenic materials.

13. The device of claim 1 wherein said one or more surface irregularities have a length or diameter within the range of about 0.005 mm to about 0.2 mm.

14. The device of claim 1 wherein said one or more surface irregularities have a height or depth of at least about 0.05 mm.

15. An embolization device for repairing vascular dysfunction, comprising:
a resilient material substrate having a first relaxed shape and a second stretched shape, wherein said first relaxed shape forms a space-filling body, and wherein said second stretched shape forms a linear body; and
one or more non-expansible porous elements positioned on said material substrate.

16. The device of claim 15 wherein said material substrate may be manufactured from at least one biologically compatible material selected from the group consisting of platinum, gold, tantalum, titanium, stainless steel, tungsten, Nitinol, shape memory alloys, polyurethane, polyvinyl alcohol, polyester, polytetrafluoroethylene, silicone, and acrylic.

17. The device of claim 15 wherein said first relaxed shape is a predetermined shape selected from the group consisting of helical, spherical, ovoidal, and conical shapes.

18. The device of claim 15 wherein said material substrate comprises at least one helical winding.

19. The device of claim 15 wherein said material substrate comprises a cylindrical structure.

20. The device of claim 15 wherein said material substrate is intermittently coiled.

21. The device of claim 15 wherein said embolization device incorporates at least one material selected from the group consisting of bio-active agents, proteins, peptides, marking agents, vascular endothelial growth factors (VEGF), basic fibroblast growth factors (bFGF), transforming growth factors-β (TGF-β) Hyaluronan derivatives [2,2], paracyclophanes, agenine-glycerine-aspartic acid (RGD), platelet derived growth factor (PDGF), thrombospondin 1 (TSP1), alginate, collagen, glycoprotein, glycosaminoglycan, endotehlial cells, tissue submucosa cells, tissue mucosa cells, and intestinal submucosa cells (SIS).

22. The device of claim 15 wherein said material substrate incorporates at least visualization element thereon.

23. The device of claim 22 wherein said visualization element is selected from the group consisting of fluoroscopic devices, x-ray devices, magnetic resonant devices, ultrasonic imaging devices, radio-opaque devices, and echogenic devices.

24. The device of claim 15 wherein said one or more porous elements are manufactured from at least one biologically compatible material selected from the group consisting of platinum, gold, tantalum, titanium, stainless steel, tungsten, Nitinol, shape memory alloys, polyurethane, polyvinyl alcohol, polyester, polytetrafluoroethylene, silicone, and acrylic.

25. A method of repairing a vascular dysfunction, comprising:
accessing a site of a vascular dysfunction in vivo;
providing an embolization device having one or more surface irregularities formed thereon;
delivering said embolization device into said vascular dysfunction; and
promoting tissue in-growth with said embolization device.

26. The method of claim 25 further comprising visualizing said embolization device within vascular dysfunction.

27. The method of claim 26 further comprising visualizing said embolization device within said vascular dysfunction with a visualization method selected from the group consisting of fluoroscopy, x-ray imaging, magnetic resonant imaging, ultrasonic imaging, radio-opaque imaging, and echogenic imaging.

28. The method of claim 29 further comprising:
providing an embolization device having a first relaxed shape and a second stretched insertion shape;
forming said embolization device into said second stretched insertion shape to facilitate the delivering said embolization device into said vascular dysfunction;
delivering said embolization device formed in said second stretched insertion shape to said vascular dysfunction; and
permitting said embolization device to return to said first relaxed shape when applied to said vascular dysfunction.

29. A method of repairing a vascular dysfunction, comprising:
accessing a site of a vascular dysfunction in vivo;
providing an embolization device comprising a material substrate having one or more porous elements positioned thereon;
delivering said embolization device into said vascular dysfunction; and
promoting tissue in-growth with said embolization device.

30. The method of claim 29 further comprising visualizing said embolization device within vascular dysfunction.

31. The method of claim 29 further comprising visualizing said embolization device within said vascular dysfunction with a visualization method selected from the group consisting of fluoroscopy, x-ray imaging, magnetic resonant imaging, ultrasonic imaging, radio-opaque imaging, and echogenic imaging.

32. The method of claim 29 further comprising:
providing an embolization device having a first relaxed shape and a second stretched insertion shape;
permitting said embolization device to return to said second stretched insertion shape to facilitate the delivering said embolization device into said vascular dysfunction;
delivering said embolization device formed in said second stretched insertion shape to said vascular dysfunction; and
permitting said embolization device to return to said first relaxed shape when applied to said vascular dysfunction.
